(19) United States
(12) Patent Application Publication
(10) Pub. No.: US 2010/0292583 A1
(43) Pub. Date: Nov. 18, 2010

(54) METHOD AND SYSTEM FOR SYNCHRONIZING BLOOD COMPONENT OR TRACE ANALYTE MEASUREMENT WITH HEART PULSE RATE

(76) Inventors: Steve C. White, La Quinta, CA (US); P. Guy Howard, Junction City, OR (US)

Correspondence Address: CHRISTIE, PARKER & HALE, LLP, PO BOX 7068, PASADENA, CA, 91109-7068, US

(21) Appl. No.: 12/779,898

(22) Filed: May 13, 2010

Related U.S. Application Data
(60) Provisional application No. 61/177,976, filed on May 13, 2009.

(51) Int. Cl. A61B 5/0205

(52) U.S. Cl. 600/483

(57) ABSTRACT

A system and method for measuring at least one blood component or trace analyte in the blood of a subject, the system including: a heart pulse rate measuring device for measuring a heart pulse rate of the subject, and a blood component or trace analyte measuring device for measuring the at least one blood component or trace analyte, wherein the blood component or trace analyte measuring device is synchronized with the heart pulse rate of the subject to perform a blood component or trace analyte measurement at a point along a measurement interval determined by the heart pulse rate.

### CROSS-REFERENCE TO RELATED APPLICATION(S)

This application claims priority to and the benefit of U.S. Provisional Patent Application No. 61/177,976 filed on May 13, 2009, in the United States Patent and Trademark Office, entitled METHOD AND SYSTEM FOR SYNCHRONIZING BLOOD COMPONENT OR TRACE ANALYTE MEASUREMENT WITH HEART PULSE RATE, the entire content of which is incorporated herein by this reference. This application contains subject matter related to U.S. Provisional Patent Application No. 61/133,892 filed on Jul. 3, 2008, entitled SYSTEM FOR NON-INVASIVE SPECTROSCOPIC DETECTION OF BLOOD ALCOHOL CONCENTRATION, the entire content of which is incorporated herein by this reference. This application contains subject matter related to U.S. patent application Ser. No. 11/945,992 filed on Nov. 27, 2007, entitled APPARATUS FOR NON-INVASIVE SPECTROSCOPIC MEASUREMENT OF ANALYTES, AND METHOD OF USING THE SAME, the entire content of which is incorporated herein in by this reference, and which claims priority to and the benefit of U.S. Provisional Patent Applications Nos. 60/949,836 and 60/966,028, the entire content of which is also incorporated herein by this reference. This application contains subject matter related to U.S. patent application Ser. No. 11/702,806, the entire content of which is incorporated herein by this reference.

### FIELD OF THE INVENTION

The present invention relates to a device and a method for synchronizing blood component or trace analyte measurement with the heart pulse rate.

### BACKGROUND OF THE INVENTION

While conducting spectroscopic blood component or trace analyte measurements on live subjects, movement of blood, due to the beating of the heart, may result in variation in the measurement of the blood component or trace analyte. Therefore, there is a need for a method and device for synchronizing blood component or trace analyte measurement with the heart pulse rate.

### SUMMARY OF THE INVENTION

An embodiment of the present invention provides a system for measuring at least one blood component or trace analyte in the blood of a subject, the system including: a heart pulse rate measuring device for measuring a heart pulse rate of the subject, and a blood component or trace analyte measuring device for measuring the at least one blood component or trace analyte, wherein the blood component or trace analyte measuring device is synchronized with the heart pulse rate of the subject to perform a blood component or trace analyte measurement at a point in a measurement interval determined by the heart pulse rate.

The heart pulse rate measuring device may include a pulse oximeter, a pressure sensor, or an electrocardiogram. A plurality of blood component or trace analyte measurements may be taken at the same point during a plurality of measurement intervals.

Another embodiment of the present invention provides a method for measuring at least one blood component or trace analyte of a subject. The method includes: measuring a heart pulse rate of the subject with a heart pulse rate measuring device, and measuring the at least one blood component or trace analyte with a blood component or trace analyte measuring device, wherein a blood component or trace analyte measurement is synchronized with the heart pulse rate of the subject at a point along a measurement interval determined by the heart pulse rate.

Another embodiment of the present invention provides an apparatus for measuring at least one blood component or trace analyte in the blood of a subject. The apparatus includes: a heart pulse rate measuring device for measuring a heart pulse rate of the subject, and a probe comprising a blood component or trace analyte measuring device for measuring the at least one blood component or trace analyte. The blood component or trace analyte measuring device is synchronized with the heart pulse rate of the subject to perform a blood component or trace analyte measurement at a point along a measurement interval determined by the heart pulse rate. The apparatus also includes a mounting support for the probe that defines at least one path of translational movement along a surface of the mounting support, and a biasing element that exerts substantially constant force to provide translational movement of the probe. The probe is translationally movable along the at least one path in a direction away from an initial point, and the probe is biased by the substantially constant force exerted by the biasing element toward the initial point.

The heart pulse rate measuring device may be located in the probe or in the mounting support for the probe.

Another embodiment of the present invention provides a method for measuring at least one blood component or trace analyte of a subject, the method includes: measuring a heart pulse rate of the subject with a heart pulse rate measuring device; providing a source of electromagnetic radiation to a probe in a housing; supporting a lower surface of at least one of a pair of adjacent fingers or toes of the subject in the housing; guiding the adjacent fingers or toes of the subject apart from one another such that the probe is positioned at an interstitial location between the fingers or toes of the subject not including the web; receiving the electromagnetic radiation reflected from the subject; and measuring the at least one blood component or trace analyte. The blood component or trace analyte measurement is synchronized with the heart pulse rate of the subject at a point along a measurement interval determined by the heart pulse rate.

### BRIEF DESCRIPTION OF THE DRAWINGS

FIG. 1 shows a flow chart of a method according to an embodiment of the present invention.

FIG. 2 shows a schematic view of a device according to an embodiment of the present invention.

FIG. 3 shows a schematic view of a device according to another embodiment of the present invention.

FIG. 4 shows a schematic view of a device according to another embodiment of the present invention.

FIG. 5 shows a graph of absorption due to pulse-added volume of arterial blood versus time as measured by a pulse oximeter and blood component or trace analyte measurement points according to an embodiment of the present invention.

FIG. 6 shows a schematic view of a device according to another embodiment of the present invention.

FIG. 7 shows a subject's hand with interstitial regions between the fingers at which measurements can be taken according to an embodiment of the present invention.

FIG. 8 shows a schematic view of a device according to another embodiment of the present invention.

FIG. 9 shows multiple pictorial views of devices according embodiments of the present invention.

### DETAILED DESCRIPTION OF THE INVENTION

The detailed description set forth below in connection with the drawings is intended as a description of embodiments of a system and method for synchronizing blood component or trace analyte measurement with heart pulse rate provided in accordance with the present invention and is not intended to represent the only forms in which the invention may be constructed or utilized. It is to be understood that the same or equivalent functions and structures may be accomplished by different embodiments that are also intended to be encompassed within the spirit and scope of the invention. As denoted elsewhere herein, like element numbers indicate like elements or features.

The spectroscopic measurement of blood components or trace analytes, such as alcohol (and its byproducts), cholesterol, and viruses, may be accomplished by way of an apparatus that transmits light (electromagnetic radiation), such as infrared light, into a test subject and analyzes the light that is reflected from the test subject. By analyzing this reflected light data, information about the blood components or trace analytes of the test subject, such as blood alcohol content, cholesterol levels, or HIV status, can be determined.

This spectroscopic measurement may be accomplished by way of a probe that is applied to a test subject in a region, which may be the interstitial region adjacent to a subject's finger or toe. The probe contains an illumination fiber or fibers that transmit light used in the spectroscopic measurement, and a return fiber or fibers that receive the light reflected by the test subject.

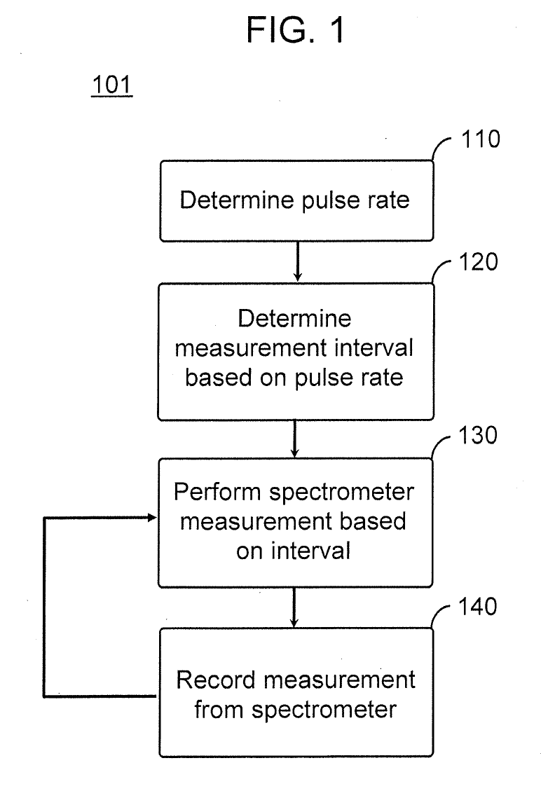

As shown in FIG. 1 according an embodiment of the present invention, a method 101 for synchronizing a blood component or trace analyte measurement with a heart pulse rate includes determining a pulse rate of a subject 110. The next step of the method 101 includes determining a measurement interval based on the pulse rate 120. Here, the measurement interval may be the same (or about the same) as the pulse rate, i.e., the interval between heart beats.

The next step of the method 101 includes performing a spectroscopic measurement of a component of the blood or of a trace analyte in the blood based on the measurement interval determined in the previous step 130. Here, the measurement may be performed at any point along the cycle of the heart beat, e.g., at the end of the contraction of the heart muscle or just before the heart muscle begins to contract. In one embodiment of the present invention, the measurement would be taken at a point along the cycle of the heart beat that would yield the measurement with the most stable and largest amplitude signal. Next, the measurement of the spectrometer will then be recorded 140.

In one embodiment of the present invention, the measurement of the blood component or trace analyte may need to be conducted only once along the cycle of the heart beat to obtain suitable data. In another embodiment of the present invention, the performing of the measurement 130 and the recording of the measurement 140 are repeated until enough measurements are recorded so that an average of the measurements will provide data with the desired accuracy. That is, multiple measurements may have to be conducted depending on subsequent data processing needs or for improving signal to noise averaging, which, in one example, would be about ten measurements. However, the performing of the measurement 130 will preferably always occur at about the same point along the cycle of the heart beat, e.g., at the end of the contraction of the heart muscle or just before the heart muscle begins to contract. Therefore, any variation (or noise) in the measurement of the spectrometer from one measurement to the next due to the movement of blood through blood vessels as a result of the heart beat may be avoided. That is, the amount of variation in measurements caused by differences in blood flow may be minimized by taking measurements at the same point in the heart beat cycle such that blood flow should be similar during each of the measurements.

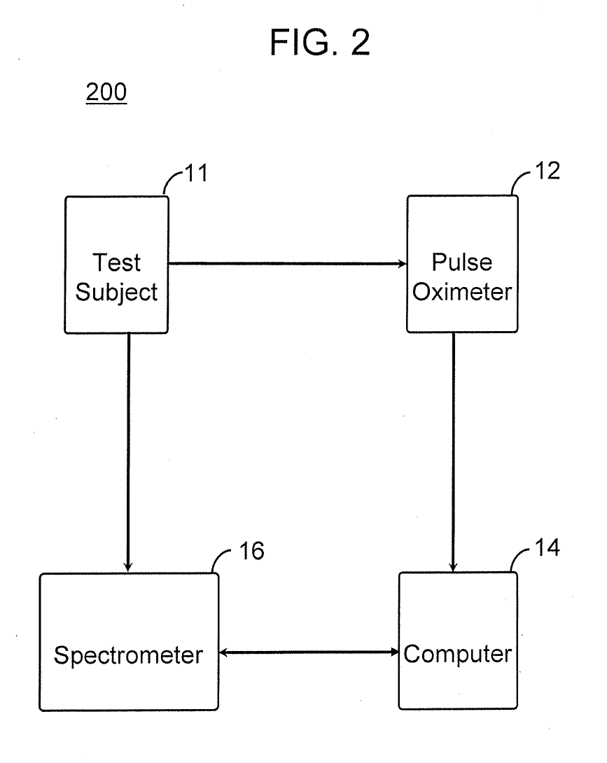

As shown in FIG. 2, according an embodiment of the present invention, a system 200 for synchronizing a blood component or trace analyte measurement with a heart pulse rate includes a pulse oximeter 12 connected to a live test subject 11 and a computer 14, and a spectrometer 16 connected to the live test subject 11 and the computer 14.

Here, the pulse oximeter 12 is used to determine the heart pulse rate of the subject 11. A pulse oximeter is a medical device that indirectly measures the oxygen saturation of a subject's blood and changes in the blood volume in the skin, and most monitors also display the subject's heart rate. A pulse oximeter is a noninvasive measurement instrument that has a pair of light-emitting diodes (LEDs) facing a photodiode through a translucent part of the subject's body, usually a fingertip, an earlobe, or the interstitial region adjacent to the finger or toe.

In an embodiment of the present invention, the heart pulse rate is measured or monitored at or near the point of measurement of the blood component or trace analyte. However, in other embodiments of the present invention, the heart pulse rate may be measured at a different point that is not near the point of measurement of the blood component or trace analyte.

One LED may be red, with a wavelength of about 660 nm, and the other LED may be infrared, with a wavelength greater than about 800 nm. Absorption of light by oxyhemoglobin and its deoxygenated form at these wavelengths differs significantly, so the oxyhemoglobin to deoxygenated hemoglobin ratio can be calculated from the ratio of the absorption of the red and infrared light. Because absorption varies in time with the heart pulse rate, the absorption due to arterial blood can be determined by subtracting the minimum absorption from the peak absorption. Further, the heart pulse rate is determined to be the interval between either minimum absorption or peak absorption readings.

Therefore, the pulse oximeter 12 may be used to determine the heart pulse rate of the subject. A computer 14 may use the heart pulse rate to determine the measurement interval of the spectrometer 16 and where along the cycle of the heart beat a measurement will be taken by the spectrometer 16. Because the blood flow rate of the subject is constantly changing, the spectrometer 16 must be able to take a measurement of the entire spectra of interest, e.g., light with wavelengths from about 2.18 μm to about 2.4 μm for the measurement of blood alcohol content, within less than about 20 ms so that blood flow rate is relatively stable or unchanged during the measurement period. However, other different wavelength ranges may be suitable for measuring blood alcohol content or other blood components or trace analytes.

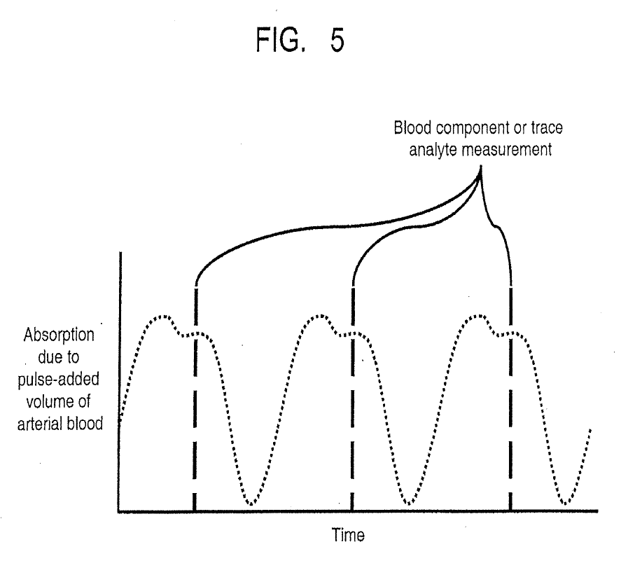

FIG. 5 shows a graph of absorption due to pulse-added volume of arterial blood versus time as measured by a pulse oximeter and blood component or trace analyte measurement times according to an embodiment of the present invention. As shown in FIG. 5, the absorption due to pulse-added volume of arterial blood varies along the cycle of the heart beat. Hence, the absorption due to pulse-added volume of arterial blood may be used to determine the heart pulse rate. Here, the blood component or trace analyte measurements are shown to occur at the same point in the cycle of the heart beat over several cycles, and the blood component or trace analyte measurements occur at relatively flat parts of the curve. Moreover, the blood component or trace analyte measurements occur in a relatively short time period so that the blood flow rate does not change much during this time period. Therefore, variability in the blood component or trace analyte measurement due to a change in blood flow rate is minimized.

In one embodiment of the present invention, a grating-based spectrometer with a line array detector is a desirable type of spectrometer. An example of a suitable spectrometer is the Ocean Optics NIRQuest 256 2.5.

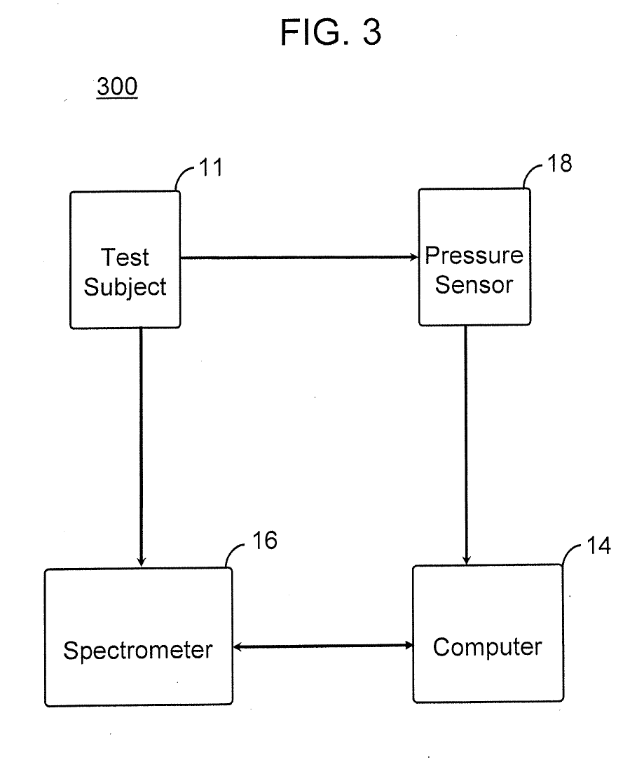

As shown in FIG. 3 according to another embodiment of the present invention, a system 300 for synchronizing a blood component or trace analyte measurement with a heart pulse rate includes a pressure sensor 18 connected to a live test subject 11 and a computer 14, and a spectrometer 16 connected to the live test subject 11 and the computer 14.

Here, the pressure sensor 18 is used to determine the heart pulse rate of the subject 10. The pressure sensor 18 measures the changes in pressure in a part of the subject, e.g., a finger, due to the cycle of the heart beat. A person of ordinary skill in the art will understand that there are various different types of pressure sensors that may be used to measure the changes in pressure associated with the cycle of the heart beat. From the changes in pressure, the heart pulse rate of the subject 11 may be determined by the computer 14. As described above with respect to the embodiment shown in FIG. 2, the computer 14 uses the heart pulse rate to determine the measurement interval of the spectrometer 16.

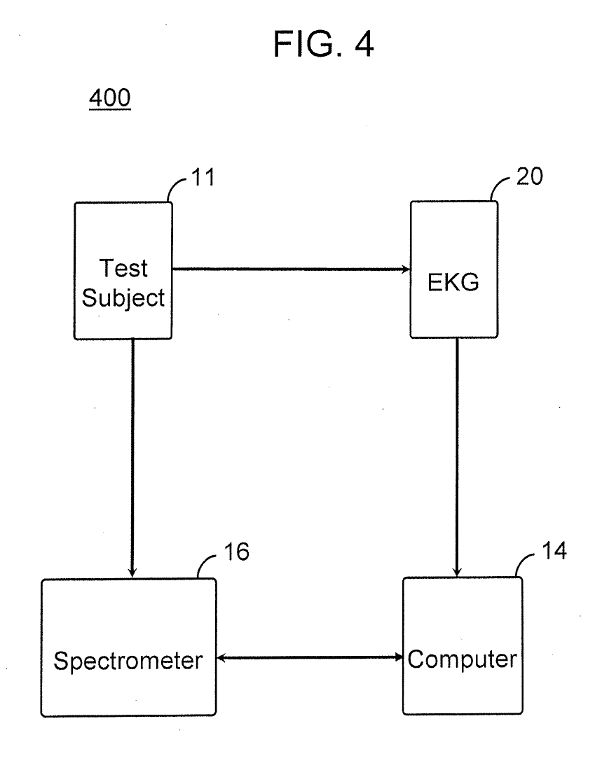

As shown in FIG. 4 according another embodiment of the present invention, a system 400 for synchronizing a blood component or trace analyte measurement with a heart pulse rate includes an electrocardiogram (EKG) 20 connected to a live test subject 11 and a computer 14, and a spectrometer 16 connected to the live test subject 11 and the computer 14.

Here, the EKG is used to determine the heart pulse rate of the subject 11 by recording of the electrical activity of the heart over time through electrodes positioned on the skin of the subject. Electrical impulses travel through the heart muscle, stimulating the muscle fibers to contract. Measuring the voltage between pairs of electrodes provides information about the electrical impulses, which are used to determine the heart pulse rate of the subject 11. As described above with respect to the embodiment shown in FIG. 2, the computer 14 uses the heart pulse rate to determine the measurement interval of the spectrometer 16.

An advantage of an embodiment of the present invention is that the system must detect the heart pulse rate of a live test subject in order to operate. Therefore, it is not possible for a user to trick the system to take a blood component or trace analyte measurement using a proxy device that does not have a heart pulse rate. For example, if the system is used in an automobile to determine that the driver does not have a blood alcohol content above a certain level before permitting the automobile to be started, the driver will not simply be able to put blood without any alcohol in the system for testing. A live driver with a heart pulse rate is required by the system to determine blood alcohol content. Therefore, the requirement of a heart pulse rate provides an advantage in that it is a security feature preventing one method of circumventing a purpose of the system.

According to an embodiment of the present invention, the spectroscopic measurement is performed at the interstitial region between a subject's fingers or toes, or the interstitial region adjacent to a single finger or toe. The interstitial regions between any two fingers or toes of the subject may also be used. In one embodiment, the interstitial region between the subject's index and middle fingers is analyzed. The housing may be large enough to accommodate a subject's single finger, two fingers, multiple fingers, or entire hand. The housing may also accommodate a subject's single toe, two toes, multiple toes, or entire foot.

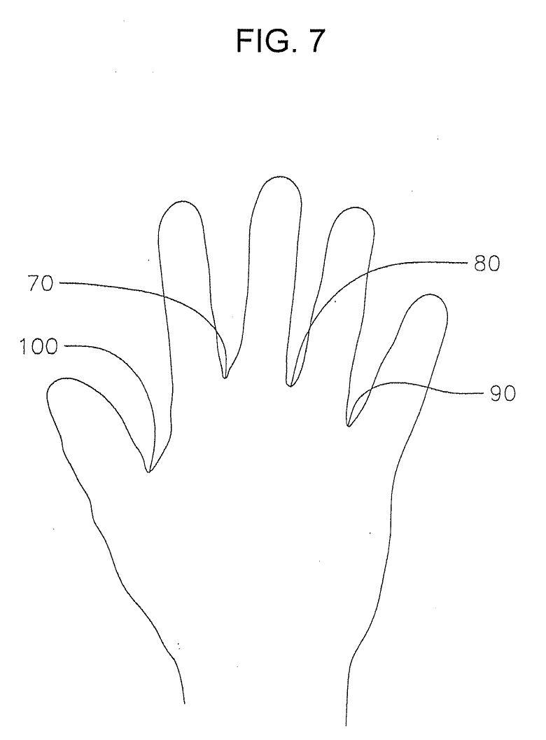

FIG. 7 shows a subject's tissue in the interstitial regions 70, 80, 90, or 100 between the subject's fingers that can used for spectroscopic measurements according to an embodiment of the present invention, e.g., the region between the base of a subject's fingers and between the plane of the subject's palm, above the web, and the top of the subject's knuckles. Advantages of measuring in the interstitial regions include low interference with the readings because the interstitial regions have relatively low density of muscle bodies, particularly in the regions between the palmar interossei and dorsal interossei muscles. Muscle bodies can contain significant variations in the concentration of lactic acid, which may interfere with the reliable detection of alcohol.

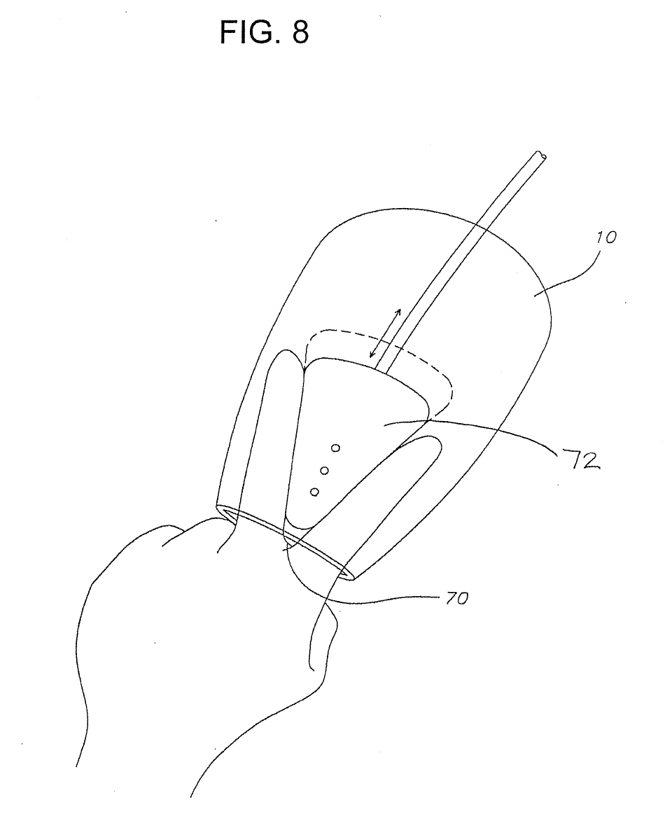

FIG. 8 illustrates an exemplary embodiment of the present invention, where a subject's interstitial region 70 being inserted into the housing 10 allowing a probe 72 to slide between the fingers and against the interstitial region 70 between the subject's fingers to obtain a spectroscopic measurement.

Another aspect of an embodiment of the present invention is directed toward a use of a single finger, toe, or digit of a subject in a simple way to simultaneously activate the apparatus for identifying the subject, scanning, and determining the blood alcohol content of the subject, and conclude the scanning.

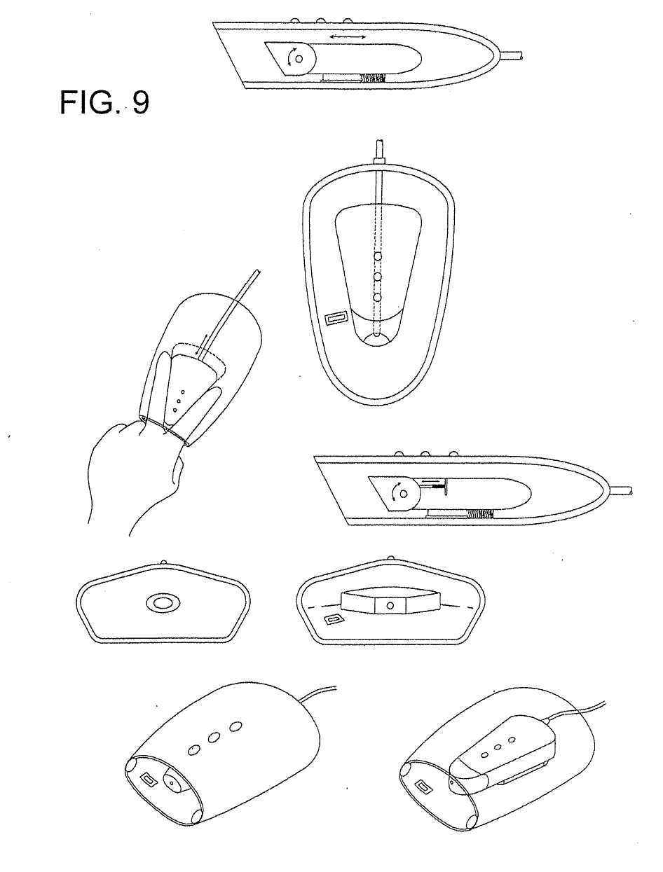

FIG. 9 shows other various embodiments of the present invention where various parts of the apparatus are designed to exert a constant pressure when applied to a subject and/or adapted to be received at an interstitial location between fingers or toes of the subject.

According to an embodiment of the present invention, both the probe and housing are smoothly contoured to enable the subject's finger, fingers, hand, toe, toes, or foot to enter the housing and press against the probe without binding, pressing, or pulling the skin in or near the region of the subject that is to be measured. Binding of the skin may create a fold or non-smooth surface of the subject's skin, which may interfere with acquisition of the signal from the subject due to variation of the subject's skin density, consistency or depth. Similarly, stretching or pulling of the subject's skin created by the probe or housing may interfere with acquisition of the signal from the subject due to variation of the subject's skin density, consistency, or depth. The smooth surface of the probe and housing minimizes the pressure and friction exerted on the subject's skin, and lessens the effect of the probe or housing pulling, pressing, stretching, or compressing the subject's skin. As a result, the pressure on the subject's skin is applied by probe in a manner that results in a reproducible pressure from the probe onto the subject's skin.

In an embodiment of the present invention, the probe is smoothly contoured with grooves on two side walls adapted to receive a first finger (or first toe) and a second finger (or second toe) of a testing subject.

The device interface with a subject may be optimized by having a probe that is moveable in one or more directions and/or subject to a constant biasing pressure on movement in those one or more directions. Accordingly, position and/or pressure sensors may be incorporated into the device, and the device may be programmed to wait until the sensors detect that the subject is in an optimal position before performing a spectroscopic measurement. In addition, parameters measured by the sensors could be stored for use in controlling activation of the device or taken into account in evaluating the results of the spectroscopic measurement.

In one embodiment, the probe has a pie configuration, where the probe is stationary mounted on the housing and biased by a substantial constant force exerted by a biasing element toward an initial testing point. In this embodiment, as the subject's interstitial region is pressed against the probe, the probe is pushed back along a path that is parallel (or substantially parallel) to the housing.

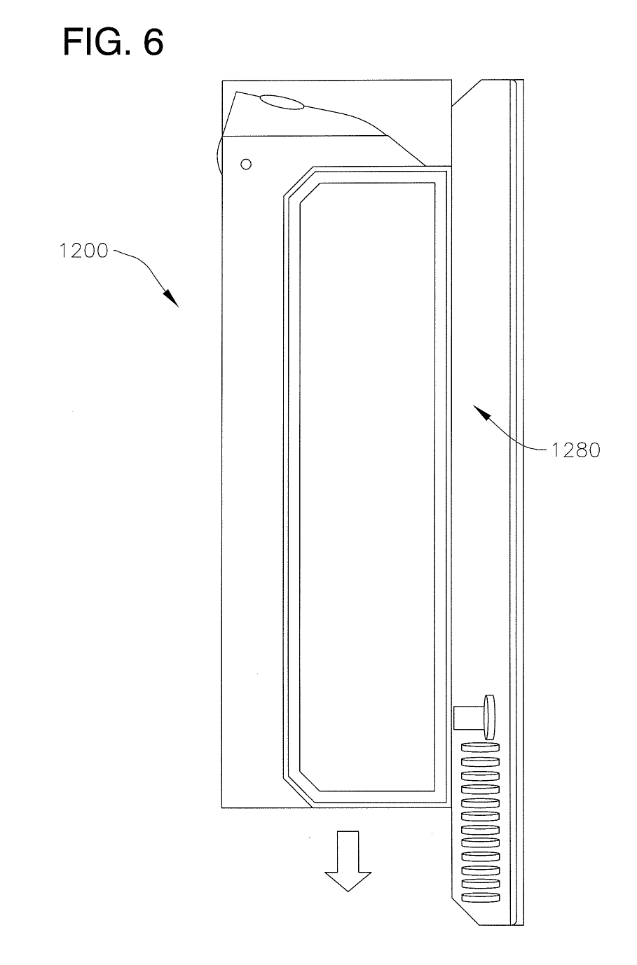

For example, FIG. 6 illustrates a probe 1200 having a translational movement according to an embodiment of the present invention. The translational movement is created along a path that is substantially parallel to a bottom supporting member 1280, where it is created by a spring mechanism, e.g., a watch-type spring, mounted toward the back of a bottom supporting member 1280.

In embodiments of the present invention, the device determining the heart pulse rate of the subject, e.g., a pulse oximeter, a pressure sensor, or an EKG, are included in either the probe or the housing. For instance, the LEDs of the pulse oximeter may be incorporated into the probe. In other examples, the pressure sensor or the EKG may be incorporated into the housing.

Although the present invention has been described through the use of exemplary embodiments, it will be appreciated by those of skill in the art that various modifications may be made to the described embodiments that fall within the scope and spirit of the invention as defined by the claims and their equivalents appended hereto. For example, aspects shown above with particular embodiments may be combined with or incorporated into other embodiments.

What is claimed is:

1. A system for measuring at least one blood component or trace analyte in the blood of a subject, the system comprising:
a heart pulse rate measuring device for measuring a heart pulse rate of the subject; and
a blood component or trace analyte measuring device for measuring the at least one blood component or trace analyte, wherein the blood component or trace analyte measuring device is synchronized with the heart pulse rate of the subject to perform a blood component or trace analyte measurement at a point in a measurement interval determined by the heart pulse rate.

2. The system of claim 1, wherein the heart pulse rate measuring device comprises a pulse oximeter.

3. The system of claim 1, wherein the heart pulse rate measuring device comprises a pressure sensor.

4. The system of claim 1, wherein the heart pulse rate measuring device comprises an electrocardiogram.

5. The system of claim 1, wherein a plurality of blood component or trace analyte measurements are taken at the same point during a plurality of measurement intervals.

6. A method for measuring at least one blood component or trace analyte of a subject, the method comprising:
measuring a heart pulse rate of the subject with a heart pulse rate measuring device, and
measuring the at least one blood component or trace analyte with a blood component or trace analyte measuring device, wherein a blood component or trace analyte measurement is synchronized with the heart pulse rate of the subject at a point along a measurement interval determined by the heart pulse rate.

7. The method of claim 6, wherein the heart pulse rate measuring device comprises a pulse oximeter.

8. The method of claim 6, wherein the heart pulse rate measuring device comprises a pressure sensor.

9. The method of claim 6, wherein the heart pulse rate measuring device comprises an electrocardiogram.

10. The method of claim 6, wherein a plurality of blood component or trace analyte measurements are taken at the same point during a plurality of measurement intervals.

11. An apparatus for measuring at least one blood component or trace analyte in the blood of a subject, the apparatus comprising:
a heart pulse rate measuring device for measuring a heart pulse rate of the subject;
a probe comprising a blood component or trace analyte measuring device for measuring the at least one blood component or trace analyte, wherein the blood component or trace analyte measuring device is synchronized with the heart pulse rate of the subject to perform a blood component or trace analyte measurement at a point along a measurement interval determined by the heart pulse rate;
a mounting support for the probe that defines at least one path of translational movement along a surface of the mounting support; and
a biasing element that exerts substantially constant force to provide translational movement of the probe,
wherein the probe is translationally movable along the at least one path in a direction away from an initial point, and the probe is biased by the substantially constant force exerted by the biasing element toward the initial point.

12. The apparatus of claim 11, wherein the heart pulse rate measuring device comprises a pulse oximeter.

13. The apparatus of claim 11, wherein the heart pulse rate measuring device comprises a pressure sensor.

14. The apparatus of claim 11, wherein the heart pulse rate measuring device comprises an electrocardiogram.

15. The apparatus of claim 11, wherein the heart pulse rate measuring device is located in the probe.

16. The apparatus of claim 11, wherein a plurality of blood component or trace analyte measurements are taken at the same point during a plurality of measurement intervals.

17. The apparatus of claim 11, wherein the heart pulse rate measuring device is located in the housing.

18. A method for measuring at least one blood component or trace analyte of a subject, the method comprising:
measuring a heart pulse rate of the subject with a heart pulse rate measuring device;
providing a source of electromagnetic radiation to a probe in a housing;
supporting a lower surface of at least one of a pair of adjacent fingers or toes of the subject in the housing;
guiding the adjacent fingers or toes of the subject apart from one another such that the probe is positioned at an interstitial location between the fingers or toes of the subject not including the web;
receiving the electromagnetic radiation reflected from the subject; and
measuring the at least one blood component or trace analyte, wherein the blood component or trace analyte measurement is synchronized with the heart pulse rate of the subject at a point along a measurement interval determined by the heart pulse rate.

19. The method of claim 18, wherein the heart pulse rate measuring device comprises a pulse oximeter.

20. The method of claim 18, wherein the heart pulse rate measuring device comprises a pressure sensor.

21. The method of claim 18, wherein the heart pulse rate measuring device comprises an electrocardiogram.

22. The method of claim 18, wherein the heart pulse rate measuring device is located in the probe.

23. The method of claim 18, wherein a plurality of blood component or trace analyte measurements are taken at the same point during a plurality of measurement intervals.

24. The method of claim 18, wherein the heart pulse rate measuring device is located in the housing.
